SEQUENCE LISTING (3 sequences)

SEQ ID NO: 1 - protein, 381 aa, Homo sapiens
  REGION 1..381: note = prosurfactant protein B (Pro-SFTPB)
MAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALAVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 2 - protein, 400 aa, Homo sapiens
  REGION 1..400: note = cytokeratin-19-fragment (Cyfra21-1)
MTSYSYRQSSATSSFGGLGGGSVRFGPGVAFRAPSIHGGSGGRGVSVSSARFVSSSSSGAYGGGYGGVLTASDGLLAGNEKLTMQNLNDRLASYLDKVRALEAANGELEVKIRDWYQKQGPGPSRDYSHYYTTIQDLRDKILGATIENSRIVLQIDNARLAADDFRTKFETEQALRMSVEADINGLRRVLDELTLARTDLEMQIEGLKEELAYLKKNHEEEISTLRGQVGGQVSVEVDSAPGTDLAKILSDMRSQYEVMAEQNRKDAEAWFTSRTEELNREVAGHTEQLQMSRSEVTDLRRTLQGLEIELQSQLSMKAALEDTLAETEARFGAQLAHIQALISGIEAQLGDVRADSERQNQEYQRLMDIKSRLEQEIATYRSLLEGQEDHYNNLSASKVL

SEQ ID NO: 3 - protein, 287 aa, Homo sapiens
  REGION 1..287: note = carcinoembryonic antigen (CEA)
GLHNHHPIKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSADVPKPSISSNNSKPVEDKDAVAFHCEPEAQNTTYLWWVNGQSLPVSPRLQLSNGNRTLTLFNVTRNDARAYVCGIQNSVSANRSDPVTLDVLYGPDTPIISPPP

(19) United States
(12) Patent Application Publication
(10) Pub. No.: US 2024/0385194 A1
(43) Pub. Date: Nov. 21, 2024

(54) METHOD FOR DIAGNOSING LUNG CANCER

(71) Applicant: Hangzhou Guangkeande Biotechnology Co., Ltd., Hangzhou (CN)

(72) Inventors: Junli GAO, Hangzhou (CN); Hong GUAN, Hangzhou (CN); Mingjie TONG, Hangzhou (CN); Junshun GAO, Hangzhou (CN); Xiaojun PENG, Hangzhou (CN); Weixin WANG, Hangzhou (CN)

(21) Appl. No.: 18/657,092

(22) Filed: May 7, 2024

(30) Foreign Application Priority Data
May 11, 2023 (CN) 2023105286767
May 15, 2023 (CN) 2023105433569
May 31, 2023 (CN) 202310631059X

(51) Int. Cl. G01N 33/574

(52) U.S. Cl. CPC G01N 33/57423; G01N 2800/56

(57) ABSTRACT

The present invention provides a biomarker: Cyfra21-1, CEA, CA125, or Pro-SFTPB, which can be used for distinguishing whether a lung cancer patient suffers from a primary lung cancer or a metastatic lung cancer, and can also be used for determining whether a primary lung cancer has metastasized.

### CROSS-REFERENCE TO RELATED APPLICATIONS

This application claims priority to the Chinese Patent Applications: Application No.: 2023105286767, filed on May 11, 2023; Application No.: 2023105433569, filed on May 15, 2023;and Application No.: 202310631059. X, filed on May 31, 2023, all of which are part of the present invention.

### REFERENCE TO AN ELECTRONIC SEQUENCE LISTING

The content of the electronic sequence listing (0256-0279PUS1.xml; Size: 5,905 bytes; and Date of Creation: Aug. 5, 2024) is herein incorporated by reference in its entirety.

### BACKGROUND OF THE INVENTION

### Field of the Invention

The invention relates to the field of medicine, specifically to use of proteomics to screen a biomarker for lung cancer and use of the biomarker in diagnosing lung cancer. In particular, it relates to a new biomarker for distinguishing whether a lung cancer is primary or metastatic and use thereof, as well as a biomarker for distinguishing whether a primary lung cancer has metastasized or not.

### Description of the Related Art

Proteomics is a scientific field dedicated to investigating the composition, location, changes, and interactions of proteins within cells, tissues, and organisms. It encompasses the study of protein expression patterns and functional profiles. The emergence of liquid chromatography-mass spectrometry (LC-MS/MS), facilitated by advancements in mass spectrometry technology, has greatly contributed to proteomics research. LC-MS/MS has become a crucial tool in this field. The development of proteomics carries significant importance in various areas, such as the search for disease diagnostic markers, drug target screening, toxicology research, and more. As a result, it finds wide application in medical research.

Lung cancer is one of the most common malignant tumors in clinics, with a high degree of malignancy and a rapid course of disease. Its prevalence and mortality rates rank first among malignant tumors, showing a rising trend year by year. The data published by the National Health Commission shows that lung cancer is a leading cause of death from malignant tumors in China, and accounts for 20% or more of all malignant tumors.

An accurate diagnosis of lung cancer is key to reducing mortality, but currently, no effective diagnostic method is available. 70% or more of patients with lung cancer have missed an optimal treatment opportunity when diagnosed. At present, there are mainly two methods of histology and imaging for diagnosing lung cancer. But the two methods have certain limitations. Since immunology and molecular biology develop, a tumor-associated protein marker shows more and more important clinical value in diagnosis and treatment of lung cancer, and has become an indispensable biological indicator for auxiliary diagnosis, observation of efficacy, and judgment of prognosis.

Resection of the primary tumor is an essential treatment means for the cure of lung cancers, but surgery itself may also promote postoperative recurrence by inducing perioperative micrometastasis dissemination, clearing anti-angiogenic signals from the tumor, inducing secretion of tumor growth factors, and inducing postoperative cell-mediated immunosuppression. Therefore, reducing the activity of tumor cells in micro-metastatic lesions and early intervention with neoadjuvant therapy has become an attractive treatment strategy, which can improve the complete control rate of tumor patients before surgery, and greatly improve their long-term survival and cure rates.

A plurality of tumor markers for the diagnosis of lung cancer, pathological typing, clinical staging, and judgment of prognosis and efficacy have been found clinically, but the diagnosis efficiency of the currently common markers (CEA and CA125) for lung cancer is not ideal. A specific tumor marker has not been found to have a higher sensitivity and specificity to diagnosis of lung cancer.

Therefore, it is of important clinical value to find a new related marker for diagnosis of lung cancer, combine a plurality of markers, and use a suitable prediction model for diagnosis of lung cancer.

### BRIEF SUMMARY OF THE INVENTION

In response to the problems in the prior art, the invention provides a biomarker for lung cancer detection, screening out a series of novel biomarkers that can early predict the risk of a lung cancer. In particular, the biomarker can distinguish whether a lung cancer patient suffers from a primary lung cancer or a metastatic lung cancer. In addition, it can effectively distinguish between the primary lung cancer and the metastatic lung cancer, so that effective treatment can be provided for different pathogenic mechanisms. Especially for the primary lung cancer, it is also possible to predict or determine in advance whether the primary lung cancer will metastasize, which has positive implications for surgical resection and prognosis.

The invention discloses a method and a kit for early detection of lung cancers. The method and the kit are used for determining multiple biomarkers included in body fluid samples obtained from subjects. The combined analysis of at least four biomarkers: CEA, CA125, CYFRA21-1, and Pro-SFTPB, offers high accuracy in the diagnosis of lung cancer when screening cohorts with known lung cancer status (primary or metastasis). This approach facilitates to determine the likelihood of primary lung cancer to metastasize, and to distinguish whether a lung cancer patient suffers from the primary lung cancer or suffers from metastatic lung cancer with tumors originated from external tissues.

On one hand, the invention provides a method for diagnosing whether a lung cancer patient suffers from a primary lung cancer or a metastatic lung cancer. The method comprises providing a body fluid sample from the lung cancer patient, testing a level of a biomarker in the body fluid sample, and distinguishing whether the lung cancer patient suffers from the primary lung cancer or the metastatic lung cancer based on the test level. The selected biomarkers include one of the following: Cyfra21-1, CEA, CA125, and Pro-SFTPB.

The invention further provides corresponding kits for determining the presence of lung cancer indicators in samples from lung cancer patients, for detecting the risk of the primary lung cancer or metastatic lung cancer in lung cancer patients, and for determining and/or quantifying the increased risk of metastasis in lung cancer patients. The kit comprises materials for measuring CEA, CA125, CYFRA21-1, and pro-SFTPB in samples.

On the other hand, the invention provides a method for diagnosing whether the primary lung cancer has metastasized, and the method comprises: providing a body fluid sample from a primary lung cancer patient, testing levels of biomarkers Cyfra21-1, CEA, CA125, and Pro-SFTPB in the samples, and distinguishing whether the primary lung cancer patient is at risk of metastasis or has metastasized based on the tested levels.

In some embodiments, biomarkers in blood samples extracted from subjects are measured. In some embodiments, the presence or absence of biomarkers in body fluid samples may be determined. In some embodiments, the levels of biomarkers in body fluid samples may be quantified.

In some embodiments, contact is made between the surface and the fluid sample. In some embodiments, a target biomarker is nonspecifically adsorbed on the surface. In some embodiments, receptors with specificity for the target biomarker are incorporated into the surface. In some embodiments, the surface binds to particles, such as beads. In some embodiments, biomarkers bind to specific receptor molecules and can determine the presence or absence of biomarker-receptor complexes. In some embodiments, the quantity of biomarker-receptor complexes may be quantified. In some embodiments, receptor molecules are linked to enzymes to facilitate detection and quantification. In some embodiments, biomarkers bind to specific relay molecules, and the biomarker-relay molecule complexes then bind to receptor molecules. In some embodiments, the presence or absence of biomarker-relay-receptor complexes can be determined. In some embodiments, the quantity of biomarker-relay-receptor complexes can be quantified. In some embodiments, receptor molecules are linked to enzymes to facilitate detection and quantification. In some embodiments, each biomarker in the body fluid sample is analyzed in sequence. In some embodiments, the body fluid sample is divided into separate sections to allow for simultaneous analysis of multiple biomarkers. In some embodiments, body fluid samples are analyzed in a single process targeting multiple biomarkers. In some embodiments, the presence or absence of biomarkers can be determined through visual inspection. In some embodiments, the quantity of biomarkers can be determined by using a spectroscopic technique. In some embodiments, the spectroscopic technique is mass spectroscopy. In some embodiments, the spectroscopic technique is UV/Vis spectroscopy. In some embodiments, the spectroscopic technique is excitation/emission technique, such as fluorescence spectroscopy. In some embodiments, the analysis of biomarkers CEA, CA125, CYFRA21-1, and Pro-SFTPB can be combined with the analysis of other biomarkers. In some embodiments, other biomarkers may be protein biomarkers. In some embodiments, other biomarkers may be non-protein biomarkers. Lung cancer patients include those suffering from the primary lung cancer and those suffering from the metastatic lung cancer, or for the primary lung cancer patients, lung cancer patients include those having metastasized and those having not metastasized. In some embodiments, additional metabolites can be introduced as needed.

In some embodiments, kits for analyzing body fluid samples are provided. In some embodiments, the kit can contain chemicals and reagents required for analysis. In some embodiments, the kit comprises a means for manipulating the body fluid sample to minimize the required operator intervention. In some embodiments, the kit can digitally record the results of analysis. In some embodiments, the kit can perform any necessary mathematical processing on the data generated from the analysis.

On one hand, the invention discloses a method for determining whether a lung cancer patient suffers from a primary lung cancer or a metastatic lung cancer, or whether the primary lung cancer is at risk of metastasis to external tissues or organs, comprising determining levels of one or more protein biomarkers and one or more metabolite markers, wherein the method comprises: obtaining body fluid samples from subjects; contacting the sample with a first reporter molecule that binds to an CEA antigen; contacting the sample with a second reporter molecule that binds to an CA125 antigen; contacting the sample with a third reporter molecule that binds to an CYFRA21-1 antigen; and making the sample come into contact with a fourth reporter molecule that binds to a pro-SFTPB antigen; wherein lung cancer patients are classified based on the levels of the first reporter molecule, second reporter molecule, third reporter molecule, and fourth reporter molecule: one category is whether the lung cancer patient suffers from the primary lung cancer or the metastatic lung cancer, and the other category is whether the primary lung cancer is at risk of metastasis.

On the other hand, the present disclosure provides a method for determining a risk of a lung cancer in a subject, comprising: obtaining body fluid samples from subjects; measuring levels of CEA, CA125, CYFRA21-1, and pro-SFTPB antigens in body fluid samples; conducting statistical analysis on the levels of CEA antigen, CA125 antigen, CYFRA21-1 antigen, and pro-SFTPB antigen in body fluid samples, determining the conditions of lung cancer patients to be primary lung cancer or metastatic lung cancer, or predicting the risk of metastasis from the primary lung cancer.

In some embodiments, the biomarkers comprise Cyfra21-1, CEA, CA125, and Pro-SFTPB.

Furthermore, the reagent is used to detect biomarkers in a body fluid sample, which includes any one of blood, urine, saliva, or sweat.

In some embodiments, the biomarkers of the invention are obtained by screening blood samples and are particularly suitable for development into blood detection reagents or kits for lung cancer prediction, especially in lung cancer patients, the biomarkers can distinguish whether the lung cancer patient or individual suffers from the primary lung cancer or the metastatic lung cancer.

Furthermore, the detection of markers in the body fluid samples refers to detecting the presence or relative abundance or concentration of biomarkers in the individual body fluid samples.

In some embodiments, relative abundance is preferred to represent the peak area of the biomarker in the detection spectrum obtained by high-performance liquid chromatography-tandem mass spectrometry. For example, if the average peak area of a biomarker measured in a control sample (individuals without lung cancer) is 500, and an average peak area measured in the lung cancer sample is 3,000, then the abundance of the biomarker in the lung cancer sample is considered six times that of the control sample.

In some embodiments, the biomarker is a combination of any two or more selected from Cyfra21-1, CEA, CA125, and Pro-SFTPB. Furthermore, a combination of Cyfra21-1, CEA, CA125, and Pro-SFTPB are included in the biomarker.

In some embodiments, the detection reagent is an antibody of the biomarker as described above, and the antibody is a monoclonal antibody.

In some embodiments, the concentration or relative abundance of the measured biomarkers in the sample are substituted into an equation to calculate whether the lung cancer patient suffers from the primary lung cancer or the metastatic lung cancer.

In some embodiments, the data analysis module includes a model equation, and the equation is:

$$Y = {\sum }_{i = 1}^{m} {K}_{i} * {X}_{i} + b$$

wherein, Y is a predictive value; i represents the i-th biomarker; m represents the number of biomarkers (m=4); Xᵢ represents the detected value of the i-th biomarker (μg/mL); Kᵢ represents the coefficient of the i-th biomarker, and b is a constant of 4.4195.

| Marker | Coefficient |
|---|---|
| Cyfra21-1 | 0.098 |
| CEA | 0.061 |
| CA125 | 0.2323 |
| Pro-SFTPB | 0.4178 |

In some embodiments, the lung cancer patient to be tested is considered as a primary lung cancer patient when the predictive value of the diagnostic model is Y≤0.518; the lung cancer patient is considered a metastatic lung cancer patient when the predictive value of the diagnostic model is Y>0.518.

The predictive value here is a standard for classifying lung cancer patients. When Y≤0.518, it indicates a high likelihood that the lung cancer patient suffers from primary lung cancer. When the predictive value of the model is Y>0.518, it indicates a high likelihood that the lung cancer patient suffers from the metastatic lung cancer. This is only one possibility and cannot be confirmed. Therefore, in terms of treatment, the method of the invention can be roughly classified, in order to provide a preliminary classification for subsequent confirmation and facilitate subsequent confirmation or treatment protocols. For example, if a lung cancer patient is considered to suffer from the primary lung cancer, direct treatment or confirmation of diagnosis for the primary lung cancer should be considered. When the lung cancer patient is considered to suffer from the metastatic lung cancer, it is necessary to check whether other body tissues or organs contain cancer cells or cancerous tissues, such as malignant nodules, malignant tumors. This kit enables early detection of cancer metastasis. After confirmation, a comprehensive treatment protocol can be formulated.

The invention further provides a device within the method, which is designed for storing the test results of biomarkers and includes a computing system comprising the aforementioned equations. The computing system computes results by inputting the test results into the equations. In some embodiments, additionally, the device comprises a data detection system and a data I/O interface; the data detection system is used to detect the biomarkers in the samples and obtain a detected value; the input interface in the data I/O interface is used to input the detected values of biomarkers. After analyzing the detected values by the data analysis module, the output interface is used to output the analysis results indicating whether the lung cancer patient has primary or metastatic lung cancer.

Another aspect of the invention provides a method for diagnosing whether a primary lung cancer individual has metastasis, comprising: providing a body fluid sample from the primary lung cancer patient, testing a level of a biomarker in the body fluid sample, determining whether the primary lung cancer has metastasis based on the level of the biomarker, wherein the biomarker is one or more of the following biomarkers: Cyfra21-1, CEA, CA125, and Pro-SFTPB

In some embodiments, the biomarker includes the combination of the following biomarkers: Cyfra21-1, CEA, CA125, and Pro-SFTPB.

In some embodiments, the body fluid sample comprises any one of blood, urine, saliva, or sweat.

In some embodiments, the biomarkers of the invention are obtained through blood sample screening and are particularly suitable for development into blood detection reagents or kits for lung cancer prediction, especially in primary lung cancer patients, the biomarkers can distinguish whether the primary lung cancer has metastasized.

In some embodiments, the biomarkers in the detected body fluid samples are to detect the presence or relative abundance or concentration of biomarkers in the individual's body fluid samples.

In some embodiments, the invention provides a system whether the primary lung cancer has metastasized, comprising a data analysis module, which is used to analyze the detected value of the biomarker in the body fluid sample, wherein the biomarker is one or more of the following: Cyfra21-1, CEA, CA125, and Pro-SFTPB.

In some embodiments, the biomarker is composed of the following biomarkers: Cyfra21-1, CEA, CA125, and Pro-SFTPB.

In some embodiments, the method of the invention also provides an equation to substitute the test results into the equation for calculation or results, and compare the results with a preset threshold to determine whether the primary lung cancer has metastasized or the level of metastasis risk. When the results are higher than the preset threshold, it is considered that the probability of primary lung cancer having metastasized is high or it is possible to metastasize. When the results are lower than the preset threshold, it is considered that the probability of primary lung cancer having metastasized is low or it is impossible to metastasize. In some embodiments, the metastasis refers to the tumor migrates to other tissues or organs other than lung.

In some embodiments, the equation is:

$$Y = {\sum }_{i = 1}^{m} {K}_{i} * {X}_{i} + b$$

wherein, Y is a predictive value; i represents the i-th biomarker; m represents the number of biomarkers (m=4); Xᵢ represents the detected value of the i-th biomarker (μg/mL); Kᵢ represents the coefficient of the i-th biomarker as shown in the table below, and b is a constant of 10.7;

| Marker | Coefficient |
|---|---|
| Cyfra21-1 | −0.1973837 |
| CEA | −0.6102073 |
| CA125 | −0.4825551 |
| Pro-SFTPB | −0.5241662 |

In some embodiments, the primary lung cancer patient to be tested is considered to suffer from no metastasis or a low probability of metastasis when the calculated result is Y≤0.525; the primary lung cancer patient to be tested is considered to suffer from metastasis or a high probability of metastasis when the model predicted result is Y>0.525. In this way, such a threshold can effectively classify the primary lung cancer, make the diagnosis more accurate, and thus specify more precise treatment protocols. For example, when the test results indicate that primary lung cancer may metastasize, it is necessary to increase monitoring of other tissues or organs during the treatment process, and the selection of treatment drugs should be targeted. Conversely, if the probability of the primary lung cancer having metastasized is low, the treatment can be focused solely on targeting lung cancer.

In the context of this invention, whether the primary lung cancer has metastasized refers to whether the primary lung cancer has metastasized to other organs or tissues, causing these organs or tissues to carry cancer cells and thus develop cancer.

In some embodiments, the method of the invention further comprises: providing a device, which can test the level of the body fluid sample, inputting the test results into the computing system automatically, which includes the computing equation described above, substituting the data of the level into the equation to obtain calculated results, comparing the calculated results with the preset threshold automatically, thus automatically outputting whether the risk of the primary lung cancer having metastasized is low or high.

In some embodiments, the Pro-SFTPB is the amino acid sequence with UniProt database No. P07988; CA125 is the amino acid sequence with UniProt database No. Q8WXI7; CEA is the amino acid sequence with UniProt database No. Q13984; Cyfra21-1 is the amino acid sequence with UniProt database No. P08727.

The beneficial effects of the invention are as follows:

Four novel biomarkers are screened to distinguish whether lung cancer patients suffer from the primary lung cancer or the metastatic lung cancer; or the biomarkers can distinguish whether the primary lung cancer will metastasize, although these four biomarkers are known, they have been found to have new applications.

Whether the primary lung cancer has metastasis refers to whether the lung cancer patient's lung cancer will metastasize to other parts of the body, for example, whether the cancer cells of lung cancer will metastasize to liver cancer, pancreatic cancer or other organs for colonization, resulting in tumorigenesis of the colonized organs.

### BRIEF DESCRIPTION OF THE DRAWINGS

FIG. 1 shows a comparison of protein markers with differential abundances_between primary lung cancer and metastatic lung cancer in lung cancer patients;

FIG. 2 shows a comparison of protein markers with differential abundances between primary lung cancer and metastatic lung cancer;

FIG. 3 shows the AUC results of the optimal combination model of the glmnet algorithm under different hyper-parameter combinations in Embodiment 2;

FIG. 4 shows the ROC curve of the lung cancer combined diagnostic model constructed in Embodiment 2 in the model group;

FIG. 5 shows the ROC curve of the lung cancer combined diagnostic model constructed in Embodiment 2 in the test group;

FIG. 6 shows the performance evaluation results of the lung cancer combined diagnostic model constructed in Embodiment 2 in the test group;

FIG. 7 shows a comparison of ROC curves of different lung cancer diagnostic models constructed in Embodiment 2;

FIG. 8 shows the AUC results of the optimal combination model of the glmnet algorithm under different hyper-parameter combinations in Embodiment 3;

FIG. 9 shows the ROC curve of the lung cancer combined diagnostic model constructed in Embodiment 3 in the model group;

FIG. 10 shows the ROC curve of the lung cancer combined diagnostic model constructed in Embodiment 3 in the test group;

FIG. 11 shows the performance evaluation results of the lung cancer combined diagnostic model constructed in Embodiment 3 in the test group;

FIG. 12 shows the ROC curves of different lung cancer diagnostic models constructed in Embodiment 3.

### DETAILED DESCRIPTION OF THE INVENTION

### Definitions:

As used herein, the term “lung cancer” refers to a malignant neoplasm in the lung, characterized by abnormal cell proliferation, wherein cell growth exceeds the growth of surrounding normal tissue and is not coordinated therewith.

As used herein, the term “lung cancer patient” refers to the mammal, preferably human beings, including those suffering from the primary lung cancer and those suffering from the metastatic lung cancer, or those suffering from the primary lung cancer having metastasized and those not suffering from the primary lung cancer having metastasized, for whom further treatment can be provided.

As used herein, the term “regression” refers to a statistical method that can specify a predictive value for a potential feature of a sample based on its observable character (or a set of observable characters). In some embodiments, this character cannot be observed directly. For example, the regression method used herein can correlate the qualitative or quantitative results of specific biomarker tests or a set of biomarker tests with the probability of a specific lung cancer patient suffering from the primary high lung cancer or the metastatic lung cancer, or the primary lung cancer having not metastasized.

As used herein, the term “sensitivity” refers to the ability to accurately identify those who suffer from diseases (i.e., true positive rate) in the context of various biochemical assays. As used herein, the term “specificity” refers to the ability to accurately identify those who suffer from no diseases (i.e., true negative rate) in the context of various biochemical assays. Sensitivity and specificity are statistical measures of binary classification tests (namely, classification functions). Sensitivity is used to quantify the avoidance of false negative, and specificity is also used to quantify the avoidance of false positive.

As used herein, “sample” refers to the presence of the biomarker to be tested, as well as its level or concentration of the test substance. The sample may be any appropriate substance according to the present disclosure, including but not limited to blood, serum, plasma, or any part thereof.

As used herein, the term “CEA” refers to carcinoembryonic antigen. As used herein, the term “CA125” refers to carcinoma antigen 125. As used herein, the term “CYFRA21-1”, also known as Cyfra 21-1, refers to the cytokeratin fragment 19, also known as the cytokeratin-19fragment. As used herein, the term “SFTPB” refers to surfactant protein B. As used herein, the term “Pro-SFTPB” refers to Pro-surfactant protein B, which is a precursor form of SFTPB.

As used herein, the term “AUC” refers to the area under the curve of the ROC plot. AUC can be used to estimate the predictive ability of a certain diagnostic test. Usually, a larger AUC corresponds to an increase in predictive ability and a decrease in the frequency of prediction errors. The AUC values may range from 0.5 to 1.0, which is a characteristic of error free prediction method.

As used herein, the term “p-value” or “p” refers to the probability that the distribution of biomarker scores for lung cancer patients with metastatic lung cancer or primary lung cancer is the same in the context of Wilcoxon rank sum test. Usually, the p-value close to zero indicates that specific statistical method will have high predictive ability when classifying subjects. As used herein, the term “CI” refers to the confidence interval, which is the interval within which a certain value can be predicted to have a certain level of confidence. As used herein, the term “95% CI” refers to the interval within which a value can be predicted to be at a confidence level of 95%.

The term “primary lung cancer” here refers to the formation of cancer or cancer cells in the lung, where there are no cancer cells in other organs of the human body. The lung is the first place or organ to produce cancer cells, resulting in the formation of malignant nodules or cancerous nodules in the lung. The malignant nodules described in the invention are different in substance from the surrounding tissue, and there is an accumulation of cancer cells in the tissue.

The so-called “metastatic lung cancer” refers to that there are no cancer cells or malignant nodules in the lung, but cancer cells or malignant nodules are generated in other organs of the human body, and with the development of cancer, they are metastasized to the lung, resulting in malignant nodules in the lung, such as pulmonary metastasis from thyroid adenocarcinoma, pulmonary metastasis from breast cancer, or pulmonary metastasis from liver cancer. These are malignant nodules in the lung, which are cancerous in the lung tissue caused by the colonization of cancer cells from other tissues in the lung.

The so-called “diagnosis or detection” in the invention refers to the detection or assay of biomarkers in the sample, or the content of the target biomarker, such as absolute or relative content, and then the presence or quantity of the target biomarker to indicate whether the individual providing the sample may have or suffer from a certain disease, or the possibility of having a certain disease. The meanings of diagnosis and detection here can be interchanged. The result of this detection or diagnosis cannot be directly used as direct result of the disease, but rather an intermediate result. If a direct result is obtained, other auxiliary methods such as pathology or anatomy are needed to confirm the presence of a certain disease. For example, the invention provides various new biomarkers associated with the lung cancer, and the changes in the content of these biomarkers are directly related to whether the lung cancer is present, whether the lung cancer patient suffers from the primary lung cancer or the metastatic lung cancer, or whether the primary lung cancer has metastasized.

The association between markers or biomarkers and lung cancer: markers and biomarkers have the same meaning in the invention. The association here refers to the direct correlation between the presence or changes in the content of a certain biomarker in the sample and a specific disease, such as a relative increase or decrease in content, indicating whether the lung cancer patient suffers from the primary lung cancer or the metastatic lung cancer, or whether the primary lung cancer has metastasized. This provides an auxiliary means for treatment and can be intervened in advance according to different situations.

The numerous biomarkers found in the serum of the invention can be used to distinguish whether the lung cancer patient suffers from the primary lung cancer or the metastatic lung cancer, or whether the primary lung cancer has metastasized. The biomarkers here can be used as individual biomarkers for direct detection or diagnosis. Choosing such biomarkers indicates a strong correlation between the relative changes in the content of these biomarkers and the type of occurrence or metastasis of lung cancer patients. Of course, it can be understood that one or more biomarkers having strong correlation with the lung cancer can be selected for simultaneous detection. It should be normally understood that in some embodiments, selecting biomarkers with strong correlation for detection or diagnosis can achieve a certain standard of accuracy, such as 60%, 65%, 70%, 80%, 85%, 90%, or 95% of accuracy, which can indicate that the intermediate value for diagnosing a certain disease can be obtained from these biomarkers, but which does not mean that the presence of a certain disease can be confirmed directly from these biomarkers. For example, when the four biomarkers of the invention are used for joint testing, lung cancer patients can be classified into primary lung cancer patients or metastatic lung cancer patients. Such classification has an accuracy of 60%-95%, which can effectively distinguish or predict the possibility of primary lung cancer having metastasized. For example, it has an accuracy of 60%-95%, which can effectively distinguish. This distinction is not absolute, but a relative probability.

Of course, differential proteins with higher ROC values can also be chosen as diagnostic markers. The so-called strong and weak are generally calculated and confirmed through some algorithms, such as contribution rate or weight analysis of the marker and lung cancer. This calculation method can be significance analysis (p-value or FDR value) and fold change. Multivariate statistical analysis mainly includes principal component analysis (PCA), partial least squares-discriminant analysis (PLS-DA), and orthogonal partial least squares-discriminant analysis (OPLS-DA), as well as other methods such as ROC analysis. Of course, other model prediction methods are also possible. When selecting specific biomarkers, the differential proteins disclosed in the invention can be selected, or other well-known biomarker combinations can be selected or combined to predict through model methods.

The model here focuses on calculation equations, which are obtained through comparative analysis. The parameter with the highest AUC is selected to obtain the most optimal model equation. The concentration of four biomarkers in the blood sample of the test patient is substituted into equation to finally obtain a result. The result is compared with the preset threshold, so that lung cancer patients can be grouped or classified based on the comparison results. For example, if the result is greater than the preset threshold, the probability of primary lung cancer with metastasis or having metastasized is considered high; otherwise, the probability of having metastasized is low. The use of the invention model is recommended to obtain data from test and validation groups. As the sample size increases, the parameters of the model can be revised to improve its predictive value.

### DETAILED DESCRIPTION

This invention will be further described in detail based on the following figures and embodiments. It should be pointed out that the following embodiments are intended to facilitate understanding of the invention and do not have any limiting effect on it. The reagents used in this embodiment are all known products obtained by purchasing commercially available products.

The invention team further conducted in-depth research on the content disclosed in the patent application (application Ser. No. 202211486610.8), and found that there were significant differences in the distribution of certain biomarkers in lung cancer patients. Through in-depth research, testing and analysis, it was found that there were significant differences in the concentration of certain biomarkers between primary lung cancer patients and metastatic lung cancer patients. At the same time, it was also found that there were significant differences in the concentration of certain biomarkers in samples of the primary lung cancer having metastasized or having not metastasized. We believe that these biomarkers cannot only distinguish between healthy individuals and lung cancer patients, but also distinguish between primary lung cancer patients and metastatic lung cancer patients, or determine whether primary lung cancer has metastasized.

Therefore, based on the eight biomarkers screened out on the basis of the patent application, four biomarkers are further screened out: Cyfra21-1, CEA, CA125, and Pro-SFTPB. The above four biomarkers have the potential to distinguish between primary lung cancer and metastatic lung cancer in lung cancer patients, or to determine whether primary lung cancer has metastasized. There are significant differences in their concentrations in some blood samples of the primary lung cancer and the metastatic lung cancer, as well as significant differences in blood samples of the primary lung cancer having metastasized or having not metastasizes. The specific embodiments below illustrate the screening process and validation of results.

### Embodiment 1 Screening Biomarkers for Lung Cancer by Proteomics

### 1. Sample Collection

Our research group collected 46 healthy controls and 85 lung cancer blood samples from August 2019 to December 2019. 36 cases suffered from the primary lung cancer having not metastasized (lung tissues were first cancerous, while other organs were not), and 49 cases suffered from the primary lung cancer having metastasized (where lung tissues were first cancerous and the lung cancer also metastasized to other tissues or organs); all enrolled patients signed an informed consent form. All lung cancer patients were confirmed by pathological examination of living tissues, and the healthy control group was normal through routine physical examination. The inclusion criteria for lung cancer patients are: (a) no history of other malignant tumors, (b) surgical treatment within one month after blood collection, and confirmed by postoperative pathology as lung cancer. The healthy individuals in the control group were selected from the physical examination center; these individuals were confirmed to have no pulmonary nodules or history of malignant tumors through chest X-ray or thin section computed tomography. After informed consent, all collected serum samples will be stored in a serum bank at −80° C.

### 2. Sample Treatment and Enzymatic Hydrolysis

Firstly, the plasma sample was centrifuged on a centrifuge for 15 minutes (15,000 g), and the supernatant was taken and filtered before performing immune-affinity chromatography to remove 14 high-abundant proteins. Then, a concentration tube with a cut-off molecular weight of 3 kDa was used to concentrate on a centrifuge (4,000 g. 1 hour). The concentrated solution was recycled and a desalting column with a cut-off molecular weight of 7 kDa was used on a centrifuge (1000×g, 2 minutes) for buffer exchange. The exchange solution was AEX-A (20 mM Tris, 4M Urea, 3% isopropanol, pH 8.0). Taking AEX-A as the blank, the protein concentration in the sample was measured by using the BCA method. According to the sample grouping in Table 1, TCEP was added to the sample and incubated at 37° C. for 30 minutes for protein reduction. Then the corresponding 10-plex TMT reagent was added and incubated at room temperature in the dark for one hour for TMT labeling reaction. Afterwards, the sample was exchanged with buffer solution using Zeba column, and the exchange solution was AEX-A. The samples labeled with 6-plex TMT were mixed and 2 mL AEX-A was added to the mixed sample, resulting in a final volume of 5.5 mL. The sample was filtered using a 0.22 m filter and the sample labeled with 10-plex TMT was separated using a 2D-HPLC system. The collected components were freeze-dried and finally Trypsin Lysine C mixed enzyme was added, incubated at 37° C. for 5 hours to hydrolyze the sample. 5 μL 10% TFA was added to terminate the enzymatic hydrolysis. A total of 60 enzymatic hydrolyzed 2D-HPLC components were used for nano-LC-MS/MS analysis.

TABLE 1
Grouping of proteomic research samples

| Sample No. | Sample grouping | TMT-6plex |
|---|---|---|
| C1 | Healthy control | 126 |
| C2 | Healthy control | 127N |
| C3 | Healthy control | 127C |
| M01 | Primary lung cancer | 128N |
| | having not metastasized | |
| M02 | Primary lung cancer | 128C |
| | having not metastasized | |
| M03 | Primary lung cancer | 129N |
| | having not metastasized | |
| MX1 | Primary lung cancer | 129C |
| | having metastasized | |
| MX2 | Primary lung cancer | 130N |
| | having metastasized | |
| MX3 | Primary lung cancer | 130C |
| | having metastasized | |
| Mix | Mixed sample | 131 |

### 3. LC-MS/MS Data Collection and Database Search Analysis

The LC-MS/MS system is a combination of Easy-nLC 1200 and Q Exactive HFX. The mobile phase A is an aqueous solution containing 0.1% formic acid and 2% acetonitrile, and the mobile phase B is an aqueous solution containing 0.1% formic acid and 80% acetonitrile. The length of the self-made analytical column is 20 cm, and ReproSil-Pur C18, 1.9μm particles from Dr. Maisch GmbH were used as the fillers. The lug peptide segment was dissolved in the mobile phase A and separated using EASY-nLC 1200 ultra-high performance liquid chromatography system. The liquid phase gradient settings: 0-26 minutes, 7% ˜22% B; 26-34 minutes, 22% ˜32% B; 34-37 minutes, 32% ˜80% B; 37-40 minutes, 80% B, maintaining liquid phase flow rate at 450 nL/min.

The peptide segments separated by the high-performance liquid chromatography system were injected into the NanoFlex ion source and atomized before being subjected to mass spectrometry analysis using Q Exactive HF-X. The ion source voltage is set to 2.1 kV, the primary mass spectrometry scanning range is set to 400-1,200, and the resolution is 60,000 (MS Resolution); the starting point of the secondary mass spectrometry scanning range is 100 m/z, and the resolution is set to 15,000 (MS2 Resolution). The TOP 20 parent ions in the data-dependent acquisition (DDA) mode sequentially enter into the HCD collision cell for fragmentation and then subjected to secondary mass spectrometry analysis. The automatic gain control (AGC) is set to 5E4, the signal threshold is set to 1E4, and the maximum injection time is set to 22 ms. To avoid repeated scanning of high-abundant peptide segments, the dynamic exclusion duration for tandem mass spectrometry analysis is set to 30 seconds.

The mass spectrometry data obtained through LC-MS/MS was retrieved using Maxquant (v1.6.15.0). The data type is TMT proteomics data based on secondary report ion quantification. The secondary spectrum used for quantification requires the proportion of parent ions in the primary spectrum greater than 75%. The database is Homo_sapiens_9606_proteome (release: 2021 Oct. 14, sequence: 20614) from Uniprot database, and common pollution databases were added to the database; contaminated proteins were removed during data analysis; the enzyme digestion method is set to Trypsin/P; the number of missed digestion sites is set to 2; the tolerances for mass errors of parent ions in First search and Main search are set to 20 ppm and 5 ppm respectively, and the tolerance for mass errors of secondary fragment ions is 20 ppm. Fixed modifications involve cysteine alkylation, while variable modifications involve oxidation of methionine and protein N-terminal acetylation. The FDRs for both protein identification and PSM identification are set to 1%.

### 4. Grouping the Samples by Orthogonal Partial Least Squares-Discriminant Analysis and Combining Significance Analysis to Screen Differential Proteins

Differential proteins were screened by using a combination of univariate analysis and multivariate statistical analysis, wherein the univariate analysis mainly includes significance analysis (p-value or FDR value) and fold change of characteristic ions in different groups, and the multivariate statistical analysis mainly includes principal component analysis (PCA), partial least squares-discriminant analysis (PLS-DA), and orthogonal partial least squares-discriminant analysis (OPLS-DA).

We found a total of 1,256 protein substances, including some newly discovered markers related to the lung cancer and some known and confirmed markers related to the lung cancer (such as carcinoembryonic antigen (CEA), carcinoma antigen 125 (CA125)).

Based on the analysis of 1,256 protein substances, protein substances with significant differences in content were obtained. All statistical analyses were conducted using R, and specific R related information is as shown in Table 2.

TABLE 2
R used in the invention and its related information

| Name | Version |
|---|---|
| R | 3.4.1 |
| Rstudio | 1.4.1717 |
| MixOmics | 6.10.9 |
| Ropls | 1.18.1 |

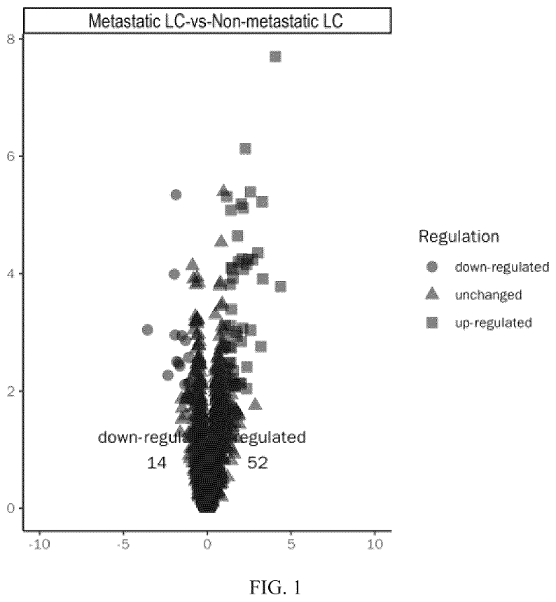

Calculating the Variable Importance for the Projection (VIP) is to measure the strength and explanatory power of the expression patterns of each protein on the classification discrimination of each group of samples. Further Wilcoxon rank sum test is performed to obtain the corrected p-value (FDR). The Wilcoxon rank results are as shown in FIG. 1. Among the 1,256 protein substances, 192 proteins were significantly elevated in the serum of patients suffering from the primary lung cancer having metastasized, and 171 proteins were significantly elevated in the serum of patients suffering from the primary lung cancer having metastasized. Compared with the patients suffering from the primary lung cancer having not metastasized, 55 proteins were significantly elevated in the serum of patients suffering from the primary lung cancer having not metastasized (see FIG. 1 for details).

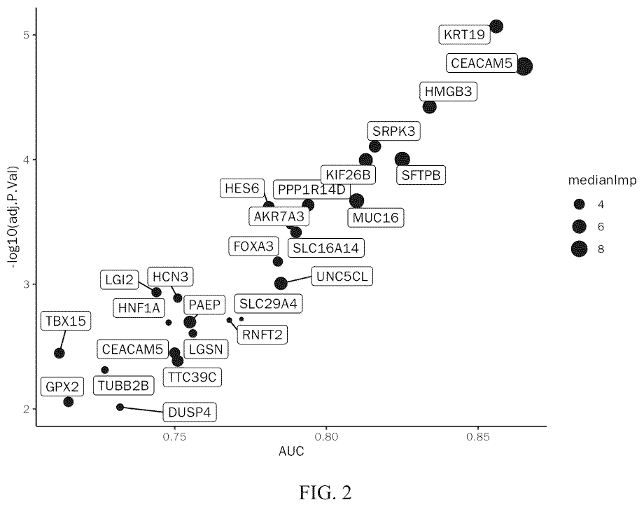

In order to find protein biomarkers that can predict whether the primary lung cancer will metastasize, we intersected the significantly elevated biomarkers in FIG. 1 and ultimately obtained 27 candidate biomarkers. Then, ROC and OPLS-DA analysis methods were used to further analyze these 27 candidate biomarkers. The results are as shown in FIG. 2, with AUC obtained from ROC analysis on the horizontal axis and VIP value obtained from OPLS-DA analysis on the vertical axis. The size of the points represents the p-value calculated from Wilcoxon test, and the color of the points represents the significance evaluation of VIP value.

According to the screening criteria for differential proteins: (1) VIP>5; (2) when FDR<0.001, i.e. VIP>5 and FDR<0.001, it is determined that there is a significant difference in protein between the two groups, and this protein is the differential protein between the two groups. According to the screening criteria, a total of four more significant differential proteins were found (pro-surfactant protein B (Pro-SFTPB), cytokerantin-19-fragment (Cyfra21-1), carcinoembryonic antigen (CEA), and carcinoma antigen 125 (CA125)).

The four main significant differential proteins found in the invention are as shown in Table 3:

TABLE 3
Differential markers of the primary lung cancer having
metastasized and the primary lung cancer
having not metastasized

| | | | UniProt database |
|---|---|---|---|
| Marker | FDR | VIP | No. |
| Prosurfactant protein B | 9.98E−05 | 7.08 | P07988 |
| (Pro-SFTPB) | | | |
| Cytokeratin-19- | 8.54E−06 | 5.79 | P08727 |
| fragment (Cyfra21-1) | | | |
| Carcinoembryonic | 1.79E−05 | 9.67 | Q13984 |
| antigen (CEA) | | | |
| Carcinoma antigen 125 | 0.000214 | 6.57 | Q8WXI7 |
| (CA125) | | | |

The smaller the FDR value and/or the larger the VIP value in Table 3, the more significant the difference between the two groups in this differential compound, and it also suggests that this differential compound may have higher diagnostic value.

Amino acid sequence (SEQ ID NO: 1) of prosur-
factant protein B (Pro-SFTPB):
MAESHLLQWLLLLLPTLCGPGTAAWTTSSLACAQGPEFWCQSLEQALQC
RALGHCLQEVWGHVGADDLCQECEDIVHILNKMAKEAIFQDTMRKFLEQ
ECNVLPLKLLMPQCNQVLDDYFPLVIDYFQNQTDSNGICMHLGLCKSRQ
PEPEQEPGMSDPLPKPLRDPLPDPLLDKLVLPVLPGALQARPGPHTQDL
SEQQFPIPLPYCWLCRALIKRIQAMIPKGALAVAVAQVCRVVPLVAGGI
CQCLAERYSVILLDTLLGRMLPQLVCRLVLRCSMDDSAGPRSPTGEWLP
RDSECHLCMSVTTQAGNSSEQAIPQAMLQACVGSWLDREKCKQFVEQHT
PQLLTLVPRGWDAHTTCQALGVCGTMSSPLQCIHSPDL
Amino acid sequence (SEQ ID NO: 2) of cyto-
keratin-19-fragment (Cyfra21-1):
MTSYSYRQSSATSSFGGLGGGSVRFGPGVAFRAPSIHGGSGGRGVSVSS
ARFVSSSSSGAYGGGYGGVLTASDGLLAGNEKLTMQNLNDRLASYLDKV
RALEAANGELEVKIRDWYQKQGPGPSRDYSHYYTTIQDLRDKILGATIE
NSRIVLQIDNARLAADDFRTKFETEQALRMSVEADINGLRRVLDELTLA
RTDLEMQIEGLKEELAYLKKNHEEEISTLRGQVGGQVSVEVDSAPGTDL
AKILSDMRSQYEVMAEQNRKDAEAWFTSRTEELNREVAGHTEQLQMSRS
EVTDLRRTLQGLEIELQSQLSMKAALEDTLAETEARFGAQLAHIQALIS
GIEAQLGDVRADSERQNQEYQRLMDIKSRLEQEIATYRSLLEGQEDHYN
NLSASKVL
Amino acid sequence (SEQ ID NO: 3) of carcino-
embryonic antigen (CEA):
GLHNHHPIKPFITSNNSNPVEDEDAVALTCEPEIQNTTYLWWVNNQSLP
VSPRLQLSNDNRTLTLLSVTRNDVGPYECGIQNELSVDHSDPVILNVLY
GPDDPTISPSYTYYRPGVNLSLSCHAASNPPAQYSWLIDGNIQQHTQEL
FISNITEKNSGLYTCQANNSASGHSRTTVKTITVSADVPKPSISSNNSK
PVEDKDAVAFHCEPEAQNTTYLWWVNGQSLPVSPRLQLSNGNRTLTLFN
VTRNDARAYVCGIQNSVSANRSDPVTLDVLYGPDTPIISPPP

### Embodiment 2: A classification model and its establishment for distinguishing lung cancer patients from primary lung cancer or metastatic lung cancer using a combination of four differential proteins

### 1. Obtaining Data

### Study Population

1,250 lung cancer patients were collected from 2020 to 2022, including 650 patients with the metastatic lung cancer and 600 patients with the primary lung cancer. All enrolled patients signed informed consent forms. All lung cancer patients were confirmed by pathological examination of living tissues, and primary lung adenocarcinoma and metastatic lung adenocarcinoma were differentiated through immune-histochemical examination. The enrolled individuals were divided into a model group (primary lung cancer n=585, metastatic lung cancer n=540) and a test group (primary lung cancer n=65, metastatic lung cancer n=60) at a ratio of 9:1.The data information is as shown in Table 4:

TABLE 4
Modeling sample information

| | Model group | Test group |
|---|---|---|
| Primary lung cancer | 585 | 65 |
| Metastatic lung cancer | 540 | 60 |

The inclusion criteria for lung cancer patients are: (a) the primary lung cancer patients have no history of other malignant tumors, and the metastatic lung cancer patients have a history of other malignant tumors, but the patients without malignant tumors in the lungs later develop lung cancer; (b) surgical treatment within one month after blood collection, and confirmed by postoperative pathology as the primary lung cancer or the metastatic lung cancer. After informed consent, all collected serum samples will be stored in a serum bank at −80° C.

This embodiment performed enzyme-linked immunosorbent assay (ELISA) on the collected serum samples to obtain the concentrations of Cyfra21-1, CEA, CA125, and Pro-SFTPB in the serum.

### 2. Statistical Analysis of Experimental Data

Shapiro Wilk's test was used to evaluate the normal distribution and non-parametric Wilcoxon test was used to analyze the differences in blood biomarker concentrations between primary lung cancer patients and metastatic lung cancer patients in the model group and test group, respectively. In the model group, a combined diagnostic model for four lung cancer biomarkers was constructed using a combination of multiple machine learning methods. The area under the curve (AUC) of the receiver operator characteristic (ROC) was estimated using a predicted probability value of 95% confidence interval (CI), in order to evaluate the discrimination ability of multivariate diagnostic models. The Youden Index (YI) is calculated by using a test group to determine the predicted probability cut-off value used to distinguish primary lung cancer patients and metastatic lung cancer patients. In addition, ROCs of individual biomarkers and different subgroups were constructed and compared. Standard descriptive statistical data was calculated, such as frequency, mean value, median, positive predictive value (PPV), negative predictive value (NPV), and standard deviation (SD), to describe the experimental results of the study population. R3.6.1 is used for statistical analysis, a p-value less than 0.05 is considered statistically significant.

### 3. Steps for Constructing a Combined Diagnostic Model for Distinguishing Primary Lung Cancer and Metastatic Lung Cancer

S101. using Cyfra21-1, CEA, CA125, and Pro-SFTPB protein markers from the samples in the model group as the original training dataset.

S102. selecting the generalized linear model (glmnet) algorithm for constructing prediction models, as well as the grid search range in the hyper-parameter optimization process of the algorithm. In this step, the grid search range for hyper-parameter optimization of each algorithm model is as shown in Table 5.

TABLE 5
Parameter grid search range of glmnet algorithm

| Algorithm | Parameter | Value |
|---|---|---|
| Generalized linear | alpha | 0.1, 0.55, 1 |
| model (glmnet) | lambda | 0.0005, 0.0055, 0.0547 |

S103. according to the algorithm and hyper-parameter setting range set in Step S102, selecting one of the hyper-parameter combination methods as the parameter for constructing a prediction model.

S104. dividing the original dataset into K subsets using a K-fold cross validation mechanism. To ensure that the proportion of majority class and minority class samples in each subset is the same as that of the original dataset, a Stratified K-Folds cross validation mechanism needs to be used for data segmentation.

S105. selecting one of the K subsets of training data obtained from Step S104 as the validation set Ddev.

S106. merging the unselected training data subsets in Step S105 to form the training data pool Dtrainl.

S107. based on the training dataset Dtrain obtained from Step S106. constructing a prediction model using the selected supervised classification algorithm and hyper-parameters.

S108. according to the prediction model obtained from Step S107. evaluating the validation set Ddev to obtain the AUC value, and storing the current prognostic prediction model with the corresponding AUC value in the prediction model pool Pool. Step S108 is to evaluate the prediction model obtained from Step S107 on the validation set determined in the current iteration, and store both the model and evaluation results in the prediction model pool for future selection of base prediction models. The evaluation mentioned in this step can be AUC value or other reasonable indicators for evaluating model performances.

S109. checking if all subsets have been validated. Step S109 is to determine whether all K subsets obtained in Step S104 have been used as validation sets and have subjected to model training. If all subsets are used as validation sets and training is completed, then proceed to Step S110; if there is a subset that has not been used as the validation set, proceed to Step S105. This step ensures that each sample in the original dataset has been validated to improve model stability and prevent from over-fitting to a subset.

S110. taking the average AUC of all models in the prediction model pool Pool as the final performance evaluation value for this combination method model, and storing the model parameters and final performance evaluation AUC values in the optimal model pool Poolbest.

S111. determining whether all hyper-parameter combinations are used to construct prediction models. Step S111 is to determine whether all algorithms and corresponding hyper-parameter combinations obtained from Step S102 have been used to construct prediction models. If all combination methods have completed the modeling, proceed to Step S112; if there is a combination method that has not completed the modeling, proceed to Step S103.

S113. selecting the model with the highest AUC value from the model set Poolbest obtained in Step S112 as the final prediction model for this biomarker combination.

### 4. Optimization Results of 4MP Model Parameters

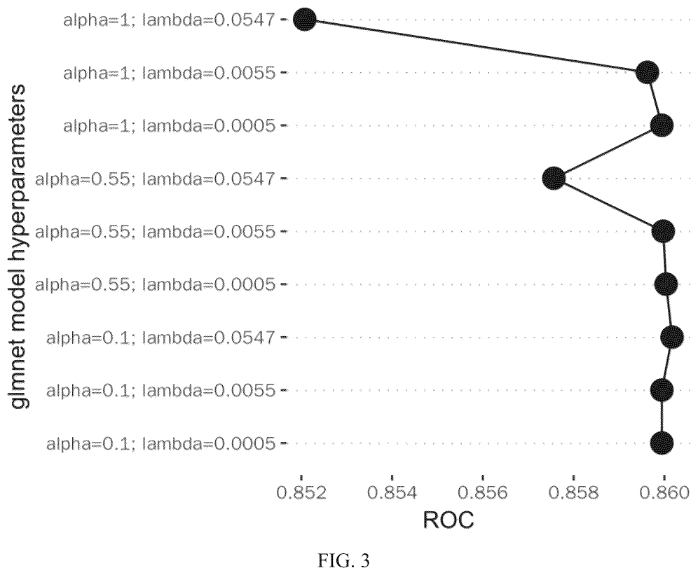

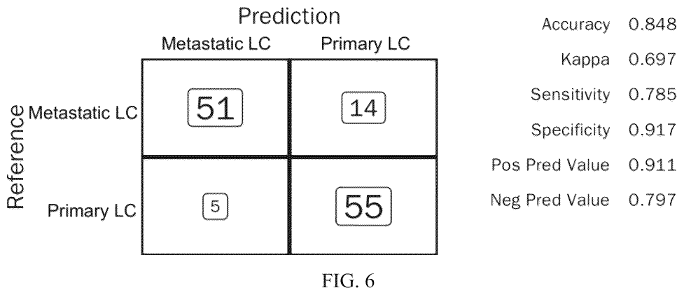

Through the above modeling steps, models constructed under 9 different combinations of glmnet algorithm hyper-parameters (FIG. 3) were obtained, and the model performance was evaluated through AUC values. As shown in FIG. 6 and FIG. 3, when the hyper-parameter combination of the glmnet algorithm is alpha=0.1 and lambda=0.0547, the AUC reaches its maximum value of 0.8602 (calculated using a 10-folds cross validation method during the modeling process).

TABLE 6
AUC of the model constructed by the glmnet algorithm
under different hyper-parameter combinations

| ALPHA | LAMBDA | AUC |
|---|---|---|
| 0.1 | 0.0547 | 0.8602 |
| 0.55 | 0.0005 | 0.8600 |
| 0.55 | 0.0055 | 0.8600 |
| 0.1 | 0.0055 | 0.8599 |
| 0.1 | 0.0005 | 0.8599 |
| 1 | 0.0005 | 0.8599 |
| 1 | 0.0055 | 0.8596 |
| 0.55 | 0.0547 | 0.8576 |
| 1 | 0.0547 | 0.8521 |

The equation for constructing a model based on the optimal hyper-parameter combination is:

$$Y = {\sum }_{i = 1}^{m} {K}_{i} * {X}_{i} + b$$

wherein, Y is a predictive value; i represents the i-th biomarker; m represents the number of biomarkers (m=4); Xᵢ represents the detected value of the i-th biomarker (μg/mL): Kᵢ represents the coefficient of the i-th biomarker (Table 7), and b is a constant of 4.4195.

TABLE 7
Coefficient of four biomarkers in the model

| Marker | Coefficient |
|---|---|
| Cyfra21-1 | 0.098 |
| CEA | 0.061 |
| CA125 | 0.2323 |
| Pro-SFTPB | 0.4178 |

### 5. Determination of Diagnostic Threshold for Lung Cancer Combined Diagnostic Model (4MP) (Distinguishing Between Primary and Metastatic)

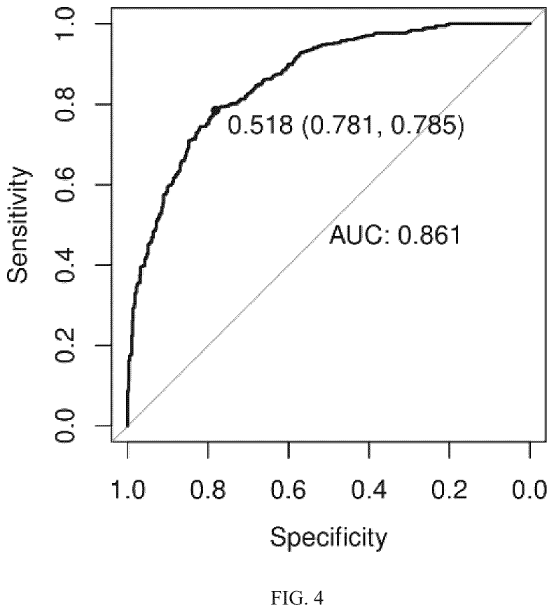

The ROC curve is drawn based on the predictive values in the model group, and the optimal diagnostic cutoff value is set to 0.518 based on the Youden index value. The lung cancer patient to be tested is considered as a primary lung cancer patient when the predictive value of the diagnostic model is ≤0.518: the lung cancer patient is considered as a metastatic lung cancer patient when the predictive value of the diagnostic model is >0.518. The results are as shown in FIG. 4: the model has an AUC of 0.861, a sensitivity of 78.5%, and a specificity of 78.1% in the model group. This is the test result obtained from the optimal calculation formula of the marker in the present invention 4. Compared to other models, the calculation structure of the formula of this model is more accurate and has a higher AUC value.

6. Validation of Lung Cancer Combined Diagnostic Model (4MP) (Distinguishing Between Primary and Metastatic Lung Cancers)

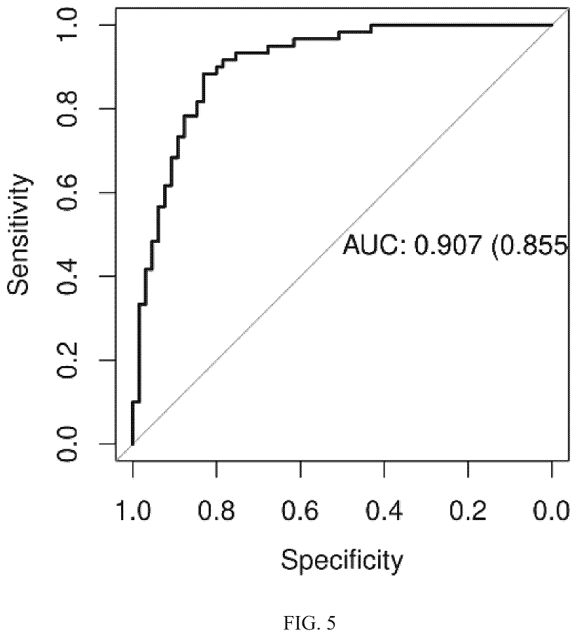

The ROC curve is drawn based on the predictive values in the test group, as shown in FIG. 5, with an AUC of 0.907. The optimal diagnostic cutoff value is set to 0.518 based on the Youden index value. That is, the lung cancer patient to be tested is considered a primary lung cancer patient when the predictive value of the diagnostic model is <0.518: the lung cancer patient to be tested is considered a metastatic lung cancer patient when the predictive value of the diagnostic model is >0.518. The results are as shown in FIG. 6: the model has an accuracy of 84.8%, a Kappa value of 0.697, a sensitivity of 78.5%, a specificity of 91.7%, a positive predictive value of 91.1%, and a negative predictive value of 79.7% in the model group.

### 7. Comparison of Diagnostic Values of Different Lung Cancer Diagnostic Models

TABLE 8
Comparison of areas under ROC curves of different diagnostic models

| PANEL | AUC | 95% CI | DIFF | P VALUE |
|---|---|---|---|---|
| 4MP | 0.907 | 0.855-0.959 | / | / |
| Pro-SFTPB | 0.849 | 0.783-0.915 | −0.057948718 | 0.017394425 |
| CYFRA21-1 | 0.723 | 0.635-0.811 | −0.184102564 | 7.19E−06 |
| CA125 | 0.617 | 0.513-0.721 | −0.290512821 | 1.04E−07 |
| CEA | 0.633 | 0.535-0.731 | −0.274102564 | 8.33E−07 |

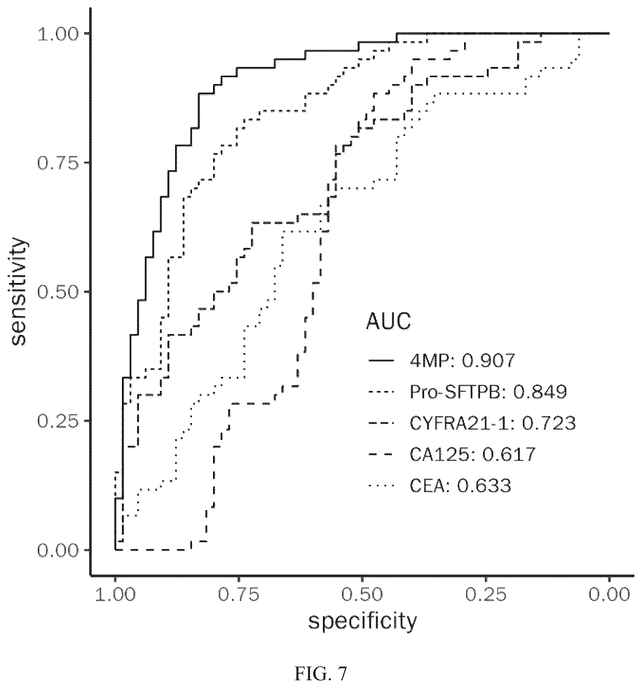

As shown in FIG. 7 and Table 8, the AUC of our model (4MP) is 0.06, 0.18, 0.29, and 0.27higher than that of a single biomarker, respectively. The DeLong's test results using the AUC significance test method showed that the diagnostic value of our model (4MP) was significantly higher (p<0.05) than that of individual biomarkers. Although the combination method can distinguish between primary and metastatic lung cancer in lung cancer patients, the above individual biomarkers also have diagnostic value and can be used separately for distinguishing between primary and metastatic lung cancer in lung cancer patients. Especially, the AUC value of Pro-SFTPB is 0.849, while the AUC value of CYFRA21-1 is 0.723.

### Embodiment 3: distinguishing whether the primary lung cancer has metastasis using a combination of four differential proteins

### 1. Sample Collection

Medical records of 740 lung cancer patients with surgical treatment at Zhejiang Cancer Hospital from May 2021 to May 2022 were collected. 474 patients with complete clinical and follow-up data were included in the model group for univariate and multivariate analyses, as well as ROC curves of actual metastasis rates. The area under the curve, specificity, and sensitivity were recorded, and the Jordan index was used as the cutoff value for judging the presence or absence of metastasis. 266 lung cancer patients were included in the test group with the model prediction ability. Sample inclusion criteria: (1) postoperative pathological staging is stage I-IIIA; (2) primary lung cancer with initial treatment, single lesion; (3) complete medical records and follow-up results. Exclusion criteria: (1) preoperative detection of multiple lung lesions or distant metastases; (2) accompanied by a history of other malignant tumors; (3) the postoperative pathology was non-small cell carcinoma; (4) due to various reasons, lymph nodes were not cleared and accurate pathological staging was not obtained after surgery; (5) death not from tumor causes; (6) incomplete medical records and follow-up results; (7) there were residual cancer cells at the cutting edge of the postoperative specimen. The data information of patients having metastasized or having not metastasized in the model group and test group is as shown in Table 9:

TABLE 9
Modeling sample information

| | Model | Test |
|---|---|---|
| | group | group |
| Primary lung cancer having not | 203 | 143 |
| metastasized | | |
| Primary lung cancer having | 271 | 123 |
| metastasized | | |

This embodiment performed enzyme-linked immunosorbent assay (ELISA) on the collected serum samples to obtain the concentrations of Cyfra21-1, CEA, CA125, and Pro-SFTPB in the serum.

### 2. Statistical Analysis of Experimental Data

Shapiro Wilk's test was used to evaluate the normal distribution and non-parametric Wilcoxon test was used to analyze the differences in blood biomarker concentrations between lung cancer patients having metastasized and having not metastasized in the model group and test group, respectively. In the model group, a combined diagnostic model for four lung cancer biomarkers was constructed using a combination of multiple machine learning methods. The area under the curve (AUC) of the receiver operator characteristic (ROC) was estimated using a predicted probability value of 95% confidence interval (CI), in order to evaluate the discrimination ability of multivariate diagnostic models. The Youden Index (YI) is calculated by using a test group to determine the predicted probability cut-off value used to distinguish primary lung cancer patients having metastasized or having not metastasized. In addition, ROCs of individual biomarkers and different subgroups were constructed and compared. Standard descriptive statistical data was calculated, such as frequency, mean value, median, positive predictive value (PPV), negative predictive value (NPV), and standard deviation (SD), to describe the experimental results of the study population. R3.6.1 is used for statistical analysis, a p-value less than 0.05 is considered statistically significant.

3. Parameter Optimization Results of the Combined Diagnostic Model (4MP) for Primary Lung Cancer having Metastasized and having not Metastasized

S101. using the concentration matrix of Cyfra21-1, CEA, CA125, and Pro-SFTPB protein markers from the samples in the model group as the original training dataset.

S102. selecting the generalized linear model (glmnet) algorithm for constructing prediction models, as well as the grid search range in the hyper-parameter optimization process of the algorithm. In this step, the grid search range for hyper-parameter optimization of each algorithm model is as shown in Table 10.

TABLE 10
Parameter grid search range of glmnet algorithm

| Algorithm | Parameter | Value |
|---|---|---|
| Generalized linear | alpha | 0.1, 0.55, 1 |
| model (glmnet) | lambda | 0.0004, 0.00404, 0.04047 |

S103. according to the algorithm and hyper-parameter setting range set in Step S102,selecting one of the hyper-parameter combination methods as the parameter for constructing a prediction model.

S104. dividing the original dataset into K subsets using a K-fold cross validation mechanism. To ensure that the proportion of majority class and minority class samples in each subset is the same as that of the original dataset, a Stratified K-Folds cross validation mechanism needs to be used for data segmentation.

S105. selecting one of the K subsets of training data obtained from Step S104 as the validation set Ddev.

S106. merging the unselected training data subsets in Step S105 to form the training data pool Dtrainl.

S107. based on the training dataset Dtrain obtained from Step S106. constructing a prediction model using the selected supervised classification algorithm and hyper-parameters.

S108. according to the prediction model obtained from Step S107. evaluating the validation set Ddev to obtain the AUC value, and storing the current prognostic prediction model with the corresponding AUC value in the prediction model pool Pool. Step S108 is to evaluate the prediction model obtained from Step S107 on the validation set determined in the current iteration, and store both the model and evaluation results in the prediction model pool for future selection of base prediction models. The evaluation mentioned in this step can be AUC value or other reasonable indicators for evaluating model performances.

S109. checking if all subsets have been validated. Step S109 is to determine whether all K subsets obtained in Step S104 have been used as validation sets and have subjected to model training. If all subsets are used as validation sets and training is completed, then proceed to Step S110; if there is a subset that has not been used as the validation set, proceed to Step S105. This step ensures that each sample in the original dataset has been validated to improve model stability and prevent from overfitting to a subset.

S110. taking the average AUC of all models in the prediction model pool Pool as the final performance evaluation value for this combination method model, and storing the model parameters and final performance evaluation AUC values in the optimal model pool Poolbest.

S111. determining whether all hyper-parameter combinations are used to construct prediction models. Step S111 is to determine whether all algorithms and corresponding hyper-parameter combinations obtained from Step S102 have been used to construct prediction models. If all combination methods have completed the modeling, proceed to Step S112; if there is a combination method that has not completed the modeling, proceed to Step S103.

S113. selecting the model with the highest AUC value from the model set Poolbest obtained in Step S112 as the final prediction model for lung cancer diagnosis.

4. Parameter Optimization Results of the 4MP Combined Diagnostic Model for Primary Lung Cancer having Metastasized and having not Metastasized

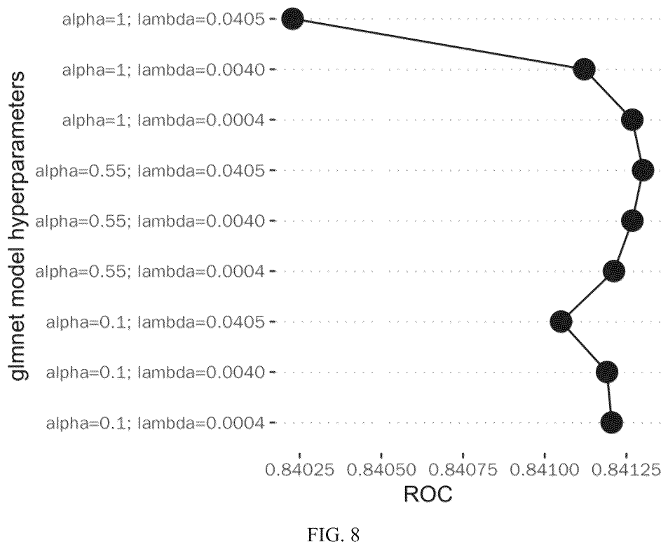

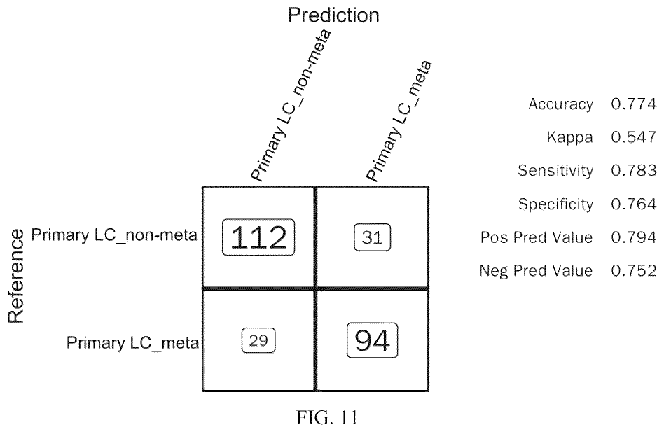

Through the above modeling steps, models constructed under 9 different combinations of glmnet algorithm hyper-parameters (FIG. 8) were obtained, and the model performance was evaluated through AUC values. As shown in FIG. 11 and FIG. 8, when the hyper-parameter combination of the glmnet algorithm is alpha=0.55 and lambda=0.040473495, the AUC reaches its maximum value of 0.8413 (calculated using a 10-folds cross validation method during the modeling process).

TABLE 11
AUC of the model constructed by the glmnet algorithm
under different hyper-parameter combinations

| ALPHA | LAMBDA | AUC |
|---|---|---|
| 0.1 | 0.000404735 | 0.841204784 |
| 0.1 | 0.004047349 | 0.841190579 |
| 0.1 | 0.040473495 | 0.841049875 |
| 0.55 | 0.000404735 | 0.841211794 |
| 0.55 | 0.004047349 | 0.841268111 |
| 0.55 | 0.040473495 | 0.841300659 |
| 1 | 0.000404735 | 0.841267973 |
| 1 | 0.004047349 | 0.841121087 |
| 1 | 0.040473495 | 0.840228489 |

The equation for constructing a model based on the optimal hyper-parameter combination is:

$$Y = {\sum }_{i = 1}^{m} {K}_{i} * {X}_{i} + b$$

wherein, Y is a predictive value; i represents the i-th biomarker; m represents the number of biomarkers (m=4); Xᵢ represents the detected value of the i-th biomarker (μg/mL); Kᵢ represents the coefficient of the i-th biomarker (Table 9), and b is a constant of 10.7.

TABLE 12
Coefficient of four biomarkers in the model

| Marker | Coefficient |
|---|---|
| Cyfra21-1 | −0.1973837 |
| CEA | −0.6102073 |
| CA125 | −0.4825551 |
| Pro-SFTPB | −0.5241662. |

5. Determination of Diagnostic Threshold for Lung Cancer Combined Diagnostic Model (4MP) (Primary Lung Cancer having Metastasized or having not Metastasized)

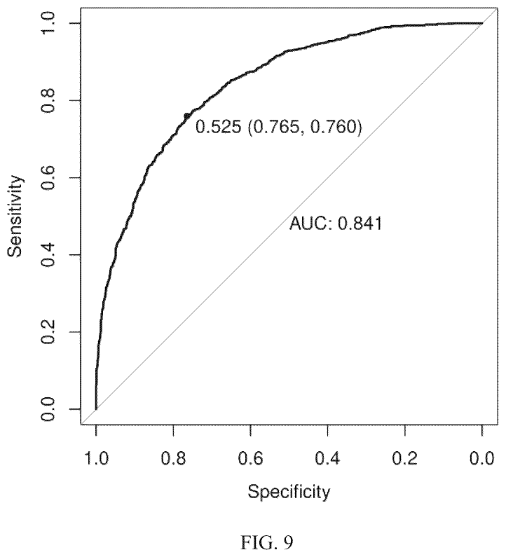

The ROC curve is drawn based on the predictive values in the model group, and the optimal diagnostic cutoff value is set to 0.525 based on the Youden index value. The primary lung cancer patient to be tested is considered to suffer from no metastasis when the predictive value of the diagnostic model is ≤0.525; the primary lung cancer patient to be tested is considered to suffer from metastasis when the predictive value of the model is >0.525. The results are as shown in FIG. 9: the model has an AUC of 0.841, a sensitivity of 76%, and a specificity of 76.5% in the model group.

6. Validation of Lung Cancer Combined Diagnostic Model (4MP) (Distinguishing Between Primary having Metastasized or having not Metastasized)

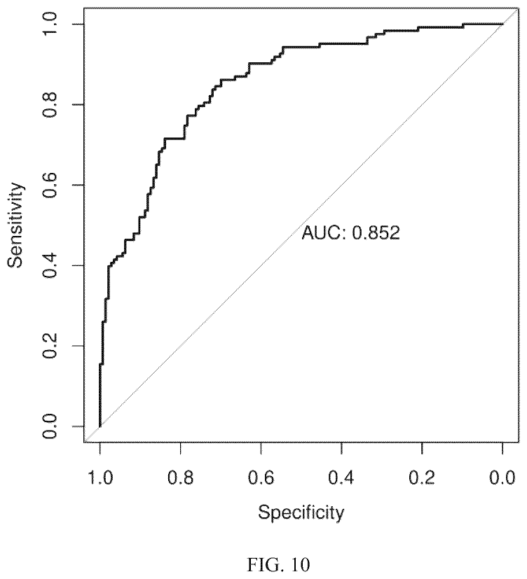

The ROC curve is drawn based on the predictive values in the test group, as shown in FIG. 10, with an AUC of 0.852. The optimal diagnostic cutoff value is set to 0.525 based on the Youden index value. The lung cancer patient to be tested is considered to suffer from no metastasis when the predictive value of the diagnostic model is ≤0.525; the lung cancer patient to be tested is considered to suffer from metastasis when the predictive value of the model is >0.525. The results are as shown in FIG. 11: the model has an accuracy of 77.4%, a Kappa value of 0.547, a sensitivity of 78.3%, a specificity of 76.4%, a positive predictive value of 79.4%, and a negative predictive value of 75.2% in the model group.

### 7. Comparison of Diagnostic Values of Different Lung Cancer Diagnostic Models

TABLE 13
Comparison of areas under ROC curves of different diagnostic models

| PANEL | AUC | 95% CI | DIFF | P VALUE |
|---|---|---|---|---|
| 4MP | 0.852 | 0.807-0.897 | / | / |
| Pro-SFTPB | 0.746 | 0.687-0.805 | −0.106543863 | 6.55E−05 |
| CYFRA21-1 | 0.59 | 0.520-0.659 | −0.262664165 | 1.23E−11 |
| CA125 | 0.68 | 0.616-0.744 | −0.172664734 | 8.56E−09 |
| CEA | 0.719 | 0.658-0.780 | −0.133321963 | 2.54E−06 |

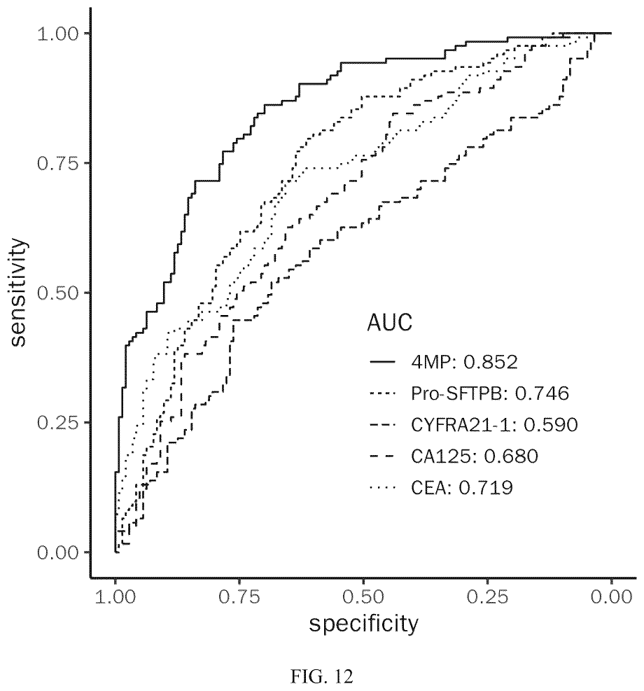

As shown in FIG. 12 and Table 13, our model (4MP) has an AUC higher than traditional single biomarkers by 0.1065, 0.263, 0.173, and 0.133, respectively. The DeLong's test results using the AUC significance test method showed that the diagnostic value of our model (4MP) was significantly higher (p<0.05) than that of traditional biomarkers or traditional biomarker combination models. However, for a single marker, it also has diagnostic value and can be used to diagnose or predict the possibility of metastasis in the primary lung cancer.

By comparing Table 13 and Table 8, it is evident that although they are all the same biomarkers, they have different meanings and diagnostic values for different classifications of lung cancer patients. From Table 13, it can be seen that the AUC value of the combined diagnosis of the four biomarkers is 0.852 for whether lung cancer patients have metastasis. With regard to whether lung cancer patients suffer from the primary or metastatic lung cancer, the AUC value of the combined diagnosis of the four biomarkers is as high as 0.907. This indicates that although they are all the same biomarkers, the classification of different lung cancers has different specificity and sensitivity, which may be determined by the different stages or directions of cancer occurrence and development in lung cancer patients. For example, in lung cancer patients who may suffer from the primary lung cancer and the metastatic lung cancer, the four biomarkers have higher sensitivity and accuracy. With regard to whether metastasis to other tissues occurs in primary lung cancer, the relative sensitivity and accuracy of the four biomarkers are low. However, the specific mechanism is not yet clear to the invention team and may be analyzed in the next step.

All patents and publications mentioned in the Description of the invention indicate that they are publicly available technologies in the art and can be used in the invention. All patents and publications referred to herein are also listed in the references as each publication is referred to separately. the invention described herein can be implemented in the absence of any one or more elements, one or more limitations, which are not specifically stated here. For example, in each instance here, the terms “including”, “substantially composed of”, and “composed of” can be replaced with the remaining two terms of either. The terms and expressions used herein are descriptive and not limited thereby, and there is no intention to indicate that these terms and explanations described herein exclude any equivalent features. However, it can be understood that any appropriate changes or modifications may be made within the scope of the invention and claims. It can be understood that the implementation examples described in the invention are all preferred implementation examples and features, and any general skilled in the art can make some modifications and variations based on the essence of the invention. These modifications and variations are also considered to fall within the scope of the invention and the scope of independent claims and dependent claims.

1. A method for diagnosing whether a lung cancer patient suffers from a primary lung cancer or a metastatic lung cancer, comprising:
providing a body fluid sample from a lung cancer patient;
testing a level of a biomarker in the body fluid sample; and
classifying the lung cancer patient based on the measured level of the biomarker to determine whether the lung cancer patient suffers from the primary lung cancer or the metastatic lung cancer,
wherein, the biomarker is one or more of the following biomarkers: Cyfra21-1, CEA, CA125, and Pro-SFTPB.

2. The method according to claim 1, wherein the biomarker comprises Cyfra21-1, CEA, CA125, and Pro-SFTPB.

3. The method according to claim 1, wherein the body fluid sample comprises blood, urine, saliva, or sweat.

4. The method according to claim 1, wherein the level of the biomarker comprises the concentration or relative abundance of the biomarker in the body fluid.

5. The method according to claim 1, wherein the biomarker consists of the following biomarkers: Cyfra21-1, CEA, CA125, and Pro-SFTPB.

6. The method according to claim 3, wherein the body fluid sample is a serum sample.

7. The method according to claim 6, wherein the lung cancer patient is considered to suffer from the primary lung cancer when the level of the biomarker exceeds a preset threshold; the lung cancer patient is considered to suffer from the metastatic lung cancer when the level of the biomarker is less than the preset threshold.

8. The method according to claim 5, wherein the tested biomarker is substituted into the following analytical equation, and a result is obtained through calculation, so as to determine whether the lung cancer patient suffers from the primary lung cancer or the metastatic lung cancer according to the calculated result; the equation is as follows:
$$Y = {\sum }_{i = 1}^{m} {K}_{i} * {X}_{i} + b$$
wherein Y is a predictive value; i represents an ith biomarker; m represents the number of biomarkers, wherein m is 4; Xᵢ represents a detected value of the ith biomarker (μg/mL); Kᵢ represents the coefficient of the ith biomarker as shown in the table below, and b is a constant of 4.4195;

| Marker | Coefficient |
|---|---|
| Cyfra21-1 | 0.098 |
| CEA | 0.061 |
| CA125 | 0.2323 |
| Pro-SFTPB | 0.4178. |

9. The method according to claim 7, wherein the lung cancer patient to be tested is considered not a primary lung cancer patient when the calculated Y value is ≤ 0.518; and the lung cancer patient is considered a metastatic lung cancer patient when the calculated Y value is >0.518.

10. The method according to claim 9, wherein the test method comprises using an antibody to bind the biomarker and obtaining the detected value of the biomarker through testing.

11. A method for diagnosing whether a primary lung cancer patient suffers from metastasis, comprising:
providing a body fluid sample from the primary lung cancer patient;
testing a level of a biomarker in the body fluid sample; and
determining whether the primary lung cancer patient suffers from metastasis based on the level;
wherein, the marker is one or more of the following biomarkers: Cyfra21-1, CEA, CA125 and Pro-SFTPB.

12. The method according to claim 11, wherein the marker consists of: Cyfra21-1, CEA, CA125 and Pro-SFTPB.

13. The method according to claim 11, wherein the marker comprises Cyfra21-1, CEA, CA125 and Pro-SFTPB.

14. The method according to claim 12, wherein the body fluid sample comprises any one of blood, urine, saliva, or sweat.

15. The method according to claim 14, wherein the blood sample is a serum sample.

16. The method according to claim 15, wherein the primary lung cancer patient is considered to suffer from metastasis or a high probability of metastasis when the level of the tested biomarker exceeds a preset threshold; the primary lung cancer patient is considered to suffer from no metastasis or a low probability of metastasis when the level of the tested biomarker is lower than the preset threshold.

17. The method according to claim 12, further comprising substituting the detected value of the biomarker into a calculation equation, and obtaining a result calculated by the equation to determine whether the primary lung cancer has metastasized through the result; wherein the equation is as follows:
$$Y = {\sum }_{i = 1}^{m} {K}_{i} * {X}_{i} + b$$
wherein Y is a predictive value; i represents an ith biomarker; m represents the number of biomarkers, wherein m is 4; Xᵢ represents the detected value of the ith biomarker (μg/mL); Kᵢ represents the coefficient of the ith biomarker as shown in the table below, and b is a constant of 10.7.

| Marker | Coefficient |
|---|---|
| Cyfra21-1 | −0.1973837 |
| CEA | −0.6102073 |
| CA125 | −0.4825551 |
| Pro-SFTPB | −0.5241662. |

18. The method according to claim 17, wherein the primary lung cancer patient to be tested is considered to suffer from no metastasis or have a low probability of metastasis when the calculated result Y is ≤0.525; and the primary lung cancer patient to be tested is considered to suffer from metastasis or have a high probability of metastasis when the predictive value Y of a model is >0.525.

19. The method according to claim 18, wherein the test method comprises using an antibody to bind the biomarker, and obtaining a detected value of the biomarker through testing.

20. The method according to claim 19, wherein the test method comprises testing a concentration of the biomarker by using an ELISA method to obtain the detected value.
